(19) United States
(12) Patent Application Publication
(10) Pub. No.: US 2006/0246093 A1
(43) Pub. Date: Nov. 2, 2006

(54) Implant analog

(75) Inventor: Stefan Ihde, Uetliburg (CH)

Correspondence Address: HUSCH & EPPENBERGER, LLC, 190 CARONDELET PLAZA, SUITE 600, ST. LOUIS, MO, 63105-3441, US

(73) Assignee: Biomed Est., Vaduz (LI)

(21) Appl. No.: 11/403,513

(22) Filed: Apr. 13, 2006

Related U.S. Application Data
(60) Provisional application No. 60/671,024, filed on Apr. 13, 2005.

(51) Int. Cl. A61K 39/08; A61C 5/00

(52) U.S. Cl. 424/239.1; 433/217.1

(57) ABSTRACT

The present invention disclosures a method of stabilizing masticatory forces during re-mineralization around dental implants. The method includes installing a dental implant and injecting botulism toxin into masticatory muscles.

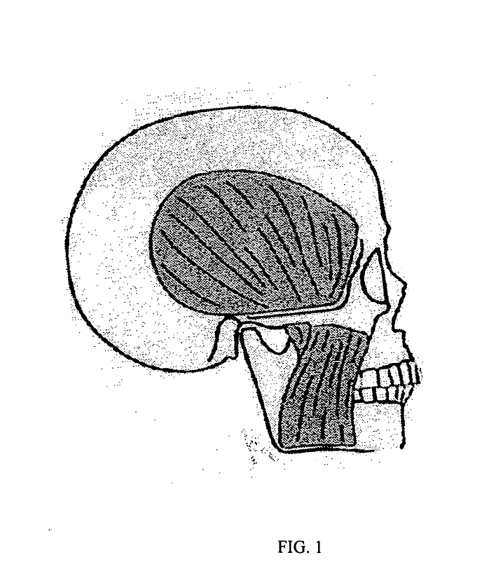

### CROSS REFERENCE TO RELATED APPLICATIONS

This application claims the benefit of U.S. Provisional Application No. 60/671,024, filed Apr. 13, 2005.

### TECHNICAL FIELD OF THE INVENTION

The invention relates generally to dental prosthetics and, more particularly, to a method of inserting such an implant.

### DESCRIPTION OF THE RELATED ART

Thanks to the use of lateral implants, almost all patients can be provided with enossally supported fixed dentures today, without any ridge augmentation procedures.

In most cases, these restorations can also be provided within treatment protocols providing for immediate loading of the implants inserted. However, patients with a significantly reduced maxillary bone supply as well as patients suspected of delivering high masticatory forces present special challenges to the implantologist and restorative therapist. Reliable control of forces reaching the bone/implant interface is particularly important in cases with limited bone supply, unfavourable vertical and sagittal dimensions or a high level of activity of the masticatory muscles.

Nevertheless, implants initially well integrated may occasionally show unexpected mobility when the bone/implant/restoration system is in actual function. There are numerous developments that may be implicated here, all of which may lead, alone or in combination, to overload of the interface between the bone and the enossal implant.

Typical causes include:

Loss of teeth or bridges in non-implanted jaw regions. This may lead to unilateral loads and subsequent overload at the bone/implant interface.
Elongation or migration of individual teeth in non-implanted jaw regions. This hazard is particularly great in the region of the mandibular third molars.
Dislocation of the temporomandibular joint following the spontaneous release of blocked muscles during the phase of functional use of the bone/implant/restoration system.
Unilateral intrusion of a bone/implant/restoration system on the side where masticatory forces are greater or bone resistance weaker, with subsequent tilting of the occlusal plane; a preferred side for chewing may develop as a consequence.
Development of atypical or at least modified masticatory patterns, resulting in a changed distribution of 1-areas and 0-areas' within the implanted jawbone. The transition of the masticatory pattern from posterior grinding movements to anterior movements exemplifies this type of development.
Loss of cortical support of isolated implants as a result of function-related morphological changes in the cranial bones.

Applicant's research demonstrates that implants showing unimpeded healing after insertion have a good chance of recovering their stability after superstructure-related overload and mobilization. It is assumed that remineralization and remodelling are possible as long as the matrix exhibits a low level of mineralization and has not changed into granulation tissue following an infection.

The primary treatment requirement in these situations is to eliminate immediately the cause of excessive loading. One way of achieving this may be to insert additional implants in the affected jaw. If the absence of sufficiently balanced occlusal surfaces in the opposing jaw is the cause, the functional masticatory balance must first be restored by restorative means. Finally, the AFMP angle (Angle fonctionelle masticatoire de Planas) will have to be adjusted in many cases. This measure will very rapidly re-establish symmetrical function. As a result, a symmetrical mineralization pattern on both sides of the jaws will gradually re-develop, once again allowing equally strong anchorage of the implant/restoration system throughout the jawbone.

Nevertheless, even regular and symmetrical masticatory forces may exceed the load threshold beyond which successful re-mineralization of the weakened direct bone-to-implant interface is no longer possible. There is a need to reduce muscular forces for a protracted period is a great advantage in this situation.

The use of lateral implants means that almost all patients can be provided with enossally supported fixed dentures today, without any ridge augmentation procedures, often within treatment protocols providing for immediate loading of the implants inserted. However, patients with a significantly reduced maxillary bone supply as well as patients suspected of delivering high masticatory forces present special challenges to the implantologist and restorative therapist.

Bone/implant/restoration systems can become mobile due to overload and underload of the peri-implant bone. This will initially result in increased mobility, without the osseointegration of the implants in the strict sense of the term being lost immediately.

Treatment strategies to decrease the loads on the bony interface, such as the use of interceptors, are not a viable alternative to the treatment described below. These devices rely on muscle-generated stimuli focused on a small number of teeth that are designed to reduce masticatory forces by inducing pain. Implants, however, do not have proprioceptors.

Splint therapies, TENS devices and similar alternative therapies are not helpful either. All these approaches are unable to deliver the required effect, which is to relieve the implant-bone interfaces of all excessive forces for a specific time (approximately 2 months).

The present invention is directed to overcoming one or more of the problems set forth above.

### REFERENCES

1. Scortecci G, Misch C, Benner K: Implants and Restorative Dentistry. London: Dunitz, 2001.
2. Ziegler C M, Haag C, Mühling J. Treatment of the recurrent temporo-mandibular joint dislocation with intramuscular botulinum toxin. Clin Oral Invest 2003; 7:52-55.
3. Daelen B, Thorwirth V, Koch A. Treatment of recurrent dislocation of the temporomandibular joint with type A botulinum toxin. Int. J. Oral Maxillofac Surg 1997; 26: 458-460.
4. Von Lindern J J, Niederhagen B, Berge S, Appel T, Reich R H. Die Behandlung der muskulären Hyperaktivität der Kaumuskulatur mit Botulinumtoxin Typ A. Dtsch Zahnärztl Z 2000; 55:26-29.
5. Hollister S J, Goldstein S A. Relationship between trabecular tissue strains and bone adaptation under controlled implant loads. In: Odgaard A, Weinans H, eds. Bone structure and remodelling (Recent advances in Human Biology vol 2). Singapore: World Scientific, 1994:51-64.
6. Rubin C T, Lanyon L E. Regulation of bone formation by applied dynamic loads. J Bone Joint Surg 1984; 66A:397-402.
7. Frost H M. The laws of bone structure, Springfield: Thomas, 1964.
8. Scully T J, Besterman G. Stress fracture—a preventable training injury. Military Medicine 1982; 147:285-287.
9. Gerber A. Okklusionslehre, Okklusionsdiagnostik und Okklusionsbehandlung im Wandel unserer Aspekte. Schweiz Monatschr Zahnmed/RMSO 1970; 80:447-470.
10. Minagi S, Akamatsu Y, Matsunaga T, Sato T J. Relationship between mandibular position and the coordination of masseter muscle activity during sleep in humans. J Oral Rehab 1998; 25:902-907.
11. Thomsen J S, Mosekilde L, Mosekilde E. Stochastic simulation of bone remodelling. In: Odgaard A, Weinans H, eds. Bone structure and remodelling (Recent advances in Human Biology vol 2). Singapore: World Scientific, 1994; 2: 167-180
12. Ihde S. Four-dimensional considerations of bone morphology and mechanics. In: Ihde S. Principles of BOI. Heidelberg: Springer, 2005:103-144.
13. Mercier P, Lafontant R. Residual alveolar ridge atrophy: Classification and influence of facial morphology. J Prosthet Dent 1979; 41:602-7.
14. Uthoff H K, Jaworski Z F G. Bone loss in response to long-term immobilization. J Bone Joint Surg 1978; 60B:420-429.
15. Ihde S. Maintenance. In: Ihde S. Principles of BOI. Heidelberg: Springer, 2005:361-387.
16. Baker L W. The influence of the forces of occlusion on the development of the bones of the skull. Int J. Orthodontia, Oral Surgery, Radiography 1922; 8:259-281.
17. Planas P. La réhabilitation neuro-occlusale. Paris: Masson (1982).
18. Chikhani L, Dichamp J, Guilbert F, Bertrand J C. Bruxisme, syndrome algodysfonctionel des articulations temporo-mandibulaires. Traitement par la toxine botulique. Etude sur 1150 cas. Implantodontie 2001; 50:27-35.
19. Ihde S. Adaptations fonctionelles de la hauteur de l'os peri-implantaire après implantation de BOI dans la mandibule. Implantodontie 2003; 12:23-33.
20. Cusack S, Cashman K D. Impact of genetic variation on metabolic response of bone diet. Proc Nutr Soc 2003; 62:901-912.
21. Minsk L, Polson A M. Dental implant outcomes in postmenopausal women undergoing homone replacement. Compendium 1998; 19:859-866.
22. Dannuci G A, Martin R B, Patterson-Buckendahl P. Ovariectomy and trabecular bone remdelling in dog. Calcified Tissue International 1987; 40:194-199.
23. Prentice A. Diet, nutrition and the prevention of osteoporosis. Public Health Nutr. 2004; 7:227-43, 1A.
24. Maki K, Miller A J, Okano T, Hatcher D, Yamaguchi T, Kobayashi H, Shibasaki Y. Cortical bone mineral density in asymmetrical mandibles: a three-dimensional quantitative computed tomography study. Eur J Orthodontics 2001; 23:217-232.
25. Ihde S. Histology of BOI implants. In: Principles of BOI. Heidelberg: Springer, 2005:307-338.

### SUMMARY OF THE INVENTION

Botulinum toxin can reduce the indirect influence of the masticatory load on the bone/enossal implant interface. If undesirable load distributions between the occlusal surfaces of both jaws are eliminated, the use of botulinum toxin on the large masticatory muscles can create an environment that is favourable to the reintegration of implants and to bone remineralization. Botulinum therapy targets the balance between demineralization and remineralization. The chances of the therapy with botulinum toxin may make it easier to decide against removing an ailing (lateral) implant and favours steps for preserving an implant/restoration system that is not entirely stable clinically due to masticatory overload.

Selected aspects of bone physiology dictate the use of botulinum toxin in recall treatment, implantological immediate-loading.

Prophylactic administration of botulinum toxin close to the time of implantation facilitates a reduction of the strength of the masseter and temporalis muscles after implantation to the point where no voluntary or involuntary movements of the masticatory system affecting the bony interface after implantation can inhibit integration of the implants.

Botulinum toxin is used therapeutically to eliminate or reduce muscular forces, even in the dental realm. Its successful use in recurring craniomandibular dislocation and hypertrophism of the masticatory muscles has been described. The well-known publications, however, emphasize the direct effect on the muscle treated rather than the indirect effect on the jawbone as stimulated to move by these muscles. The present article explains how this indirect effect can be exploited in dental implantology.

For purposes of treatment planning in dental implantology, Misch divided the quality of the bone substrate into four classes labelled D1 to D4. Bone quality at baseline depends on the prevailing nutritional and loading situation. During implantological treatment, however, it is modified by various factors, which occasionally operate synergistically. On one hand, the surfaces of enossal implants in themselves appear to corticalize the immediately surrounding bone. On the other hand, the load influences bone adaptation and the increase in density (compacting) of the trabecular bone. Masticatory forces transmitted into the bone provide additional stimulation and maturation of the bone at its interface with the implant. Ultimately, a modification in the global direction of the load within the implanted cranial bone may change masticatory function and effect changes in the mineralization pattern of the bone as a whole.

Implantological treatment protocols must take into consideration all three of these factors, both quantitatively and in their temporal sequence. The substrate of any dental implant therapy is osteonal bone, which shows a typical and invariable reaction to both trauma and changes in load: bone multicellular units (BMUs) are formed, resulting in an initial increase in porosity and ultimately in a reorientation of the osteonal architecture and in an adapted mineralization pattern. Increased numbers of BMUs appear when microcracks in bone subjected to overload are repaired; however, they also create tunnels through underloaded bone areas. The frequency of BMU activation is increased in mechanically overloaded and underloaded bone.

If enossally supported bone/implant/restoration systems are installed, the implants must be given primary stability at insertion, and adequate direct bone contact must be preserved during the bone's repair phase. At the same time, the bone must not be overloaded in any phase; this would result in a detachment of the bony interface from the implant and in an accumulation of microcracks, potentially increasing porosity to the point where the matrix structure is destroyed (post-traumatic osteoporosis). For this reason, a healing phase without any loading is obligatory for many implant systems.

Treatment protocols using immediate loading prefer implants offering sufficient macromechanical anchorage (such as compression screw implants or basal implants). At the same time, it is attempted to prosthodontically splint the numerous implants inserted in order to avoid load peaks on individual implants. Generally, cortical bone support is preferred to spongious bone support. Cortical bone is needed for skeletal support. By way of macrotrajectories, it transmits immense loads generated by the body's own weight as well as by muscular tension.

These aspects are merely illustrative of the innumerable aspects associated with the present invention and should not be deemed as limiting in any manner. These and other aspects, features and advantages of the present invention will become apparent from the following detailed description when taken in conjunction with the drawings.

### DETAILED DESCRIPTION

The present invention is a system and method for use of botulinum toxin for reduced loading of masticatory forces during a healing process after insertion of dental implants. The system includes at least one implant. In one embodiment the implant is a basal implant such as those disclosed in U.S. Pat. No. 6,402,516 B2, which is incorporated by reference herein. The system also includes a dosage association or chart. The system finally includes a means of administering the botulinum toxin, which in at least one embodiment is a hypodermic needle. In operation, the dental implant is installed. Next a dosage is calculated. Dosage calculation may take into account at least some of the following: the patient's age and overall muscle strength, the development of the masseter and/or temporomandibular muscles, the position of the dental implant, the diet of the patient, the number of implants, the location of the botulinum toxin injection, the presence of nocturnal parafunction, the amount of bone into which the implant has been installed, the nature and quality of the bone into which the implant is being installed and the like. In one embodiment of the present invention, a clinical judgment may be exercised by the dentist or surgeon in order to arrive at a dose. In another embodiment, a chart, computer memory or other look up means can be used to associate a preferred dosage with one or more of the dosage factors enumerated above. Finally, the dentist or surgeon injects the botulinum toxin into the patient's masseter and/or temporomandibular muscle. Thereafter the patient is monitored for successful healing of the implant.

### EXAMPLE 1

A 55-year-old, otherwise healthy female patient presented with a request for dental implantological treatment of her maxilla. Two solid-screw implants had already been inserted in the mandible three years previously, implants that supported a cantilevered crown block. The clinical findings were dominated by the impressively large masseter muscles bilaterally. The patient reported massive nocturnal parafunction (grinding, clenching), which sometimes almost made her unable to open her mouth in the morning. In principle, neither high masticatory forces nor parafunction is a contraindication for implantological therapy, as both these factors result in highly mineralized bone.

The patient was treated with compression-screw implants and a combination of compression-screw and lateral implants in the upper jaw. At the same time, the lower jaw was completely restored with ceramo-metal crowns and bridgework in order to optimize the three-dimensional profile of the occlusal surfaces. Each masseter muscle was medicated with 200 units of botulinum toxin (Dysport, Ipsen Pharma, 76259 Ettlingen, Germany), distributed over two sites: one dose was injected from externally at the caudal muscle attachment above the mandibular prominence angle, while the second dose was injected from intraorally into the anterior upper part of the muscle, below its attachment to the zygomatic arch.

At the four-day and four-week recall appointments, no muscle contraction was seen clinically, and the mouth opening was unchanged. The inserted implants, which had been loaded immediately, were shown to be stable and well-integrated at each follow-up. Only one of the older mandibular solid-screw implants was found to have loosened after 14 months. It was replaced by an additional lateral implant. The implant loss was not attributed to the effect of the botulinum toxin but to a lateral detachment of the bone from the vertical implant interface.

Subjectively, the patient was very happy about the effect of the medication; she reported that her jaw muscles were no longer tense when she woke up in the morning, something she considered a considerable improvement in her quality of life. As the temporal muscles had not been medicated, mastication of soft food remained possible throughout. Strength gradually returned to the muscles after three months. Muscular stiffness in the morning, by contrast, did not return.

### EXAMPLE 2

A male patient, who was 75 years old when therapy started, had received a tooth- and implant-supported circular maxillary ceramo-metal bridge six years previously. At the same time the mandible was restored, the mandibular partial denture had also been redone in order to adapt the occlusal plane, the curve of Spee and the inclinations of the cusps. Shortly after this initial therapeutic phase, the patient no longer showed up at the scheduled recall appointments. He did not reappear until after his maxillary bridge appeared to have loosened, as he himself had noticed.

Clinical examination showed that the anterior overbite had become considerably deeper, which is why the overall masticatory pattern had shifted in the direction of an anterior pattern. Undesirable lever forces resulted and may have overtaxed the regenerative capacity of the cortical bone, especially in the distal region. Possible reasons why the bite had become deeper were abrasion of the teeth of the mandibular denture, distal, “diving” of the denture, elongation of the mandibular anterior teeth and tilting of the maxillary occlusal plane due to remodeling and function-related jawbone resorption.

In this case massive mobility of implant/restoration systems may occur on severe overload even though 9 implants with 13 enossal load-transmitting disks and one natural tooth were present for load transmission. Tensile stress in the maxillary first molar region caused the most pronounced porosity and, hence, the most pronounced mobility. The large number of implants and load transmission interfaces present facilitated rapid remineralization.

The first treatment step required was to readjust the occlusal plane, which required maxillary and mandibular modifications. The remaining natural tooth 13 was removed and replaced by an additional lateral implant. However, the maxillary implants—with the exception of the newly inserted one—were visibly mobile: nor did mobility decrease after the new bridge had been inserted, not even after several weeks. However, this mobility was not painful to the patient.

As masticatory function appeared to be well adjusted, nocturnal parafunction was suspected to be the reason why implant mobility persisted, especially since the masseter and temporalis muscles appeared extremely well developed and strong in this patient. In this situation, the masseter muscles were temporarily paralyzed bilaterally using botulinum toxin (Dysport, Ipsen Pharma, 76259 Ettlingen, Germany). A total of 400 units of this preparation was injected, divided evenly between the two muscles and injected in two areas per muscle in order to get both parts of each muscle to respond. No activity of the treated muscles was noted at the four-day and at the six-week follow-up appointments.

At eight weeks, the clinical mobility of the bridge had been considerably reduced, something that can be attributed to increased bone mineralization in the implants interface region. Further stabilization of the implant/restoration system could be observed over the next twelve months following botulinum toxin therapy, despite the fact that the clinical examination of the masseter muscles at three months post-treatment had already indicated full regeneration of both muscles. The patient himself reported that he felt the bridge and implants to be more stable than ever before.

### EXAMPLE 3

A 58-year-old, otherwise healthy male patient had to have several maxillary and mandibular teeth extracted, which was done in a single session. At the same time, six enossal implants were inserted (four maxillary, two mandibular). The maxilla and the mandible were restored with long-term temporary bridges according to the immediate-loading protocol for lateral implants. After twelve months it was noted that the maxillary long-term temporary bridge was noticeably mobile even though all implants were firmly connected with the bridge. Premature contacts between the two bridges had appeared in the posterior region. Discrete radiolucent regions were seen around all basal disks in the maxilla, which was interpreted as signalling the presence of overload-related osteolysis.

Since only four implants had initially been inserted in the maxilla even though there was room for more implants, a combination surgical/drug treatment approach was selected prior to fabricating the definitive restorations. Four additional lateral implants were inserted and combined with the existing implants using a circular bridge. On the same occasion, the occlusion was thoroughly adjusted as well, especially in the mandible.

At the time the new maxillary bridge, a total of 400 units of botulinum toxin were injected bilaterally into the masseter muscles. No activity of either masseter muscle was noted at all at the one-week and six-week follow-up appointments. Muscular strength returned after four months without adversely affecting the stability of the implant/restoration system. No pathological findings were found at any of the recalls within two years after the developments described.

The second surgical intervention created an additional impetus for comprehensive remodelling, which is why the entire jawbone was once again pervaded by osteons, this time more successfully and more thoroughly. Due to their greater stability the four new implants may bear by far the largest part of the entire masticatory functional load in a situation in which slightly mobile implants are rigidly connected with new implants exhibiting primary stability using an immediate-loading protocol. This situation tends not to favour the integration of the added implants, something that might constitute an indication for the therapeutic use of botulinum toxin.

Discussion

Comprehensive insertion of enossal implants and immediately loaded restorations will change all parameters of masticatory function; the newly created occlusal surfaces will be included in the masticatory process. This results in considerable changes in the patterns of muscular function, which in turn influence the morphology of the jawbone and thus the relative position of the arches.

This factor alone requires comprehensive occlusal adjustment by subtractive or additive means. Also, pre-operative muscle blocks do not resolve until after several months. These, too, result in changed jaw relationships and can in themselves cause overload at the bone/implant interface.

Most patients are able to move their dental arches congruently during the day (voluntary movement). During phases in which voluntary control is absent, that is, especially during the night, the jaws can meet in positions that greatly deviate from their daytime positions. If this happens, balance is lost. Muscular dynamics during the patient's sleep are unique and differ from those during voluntary clenching; they exert a greater mechanical load on the temporomandibular joint on the balancing side.

The administration of botulinum toxin reduces the risk that gradual or sudden changes in mandibular position damage the bony interface of immediately loaded implants before they are detected at the scheduled recall sessions. In early phases of the therapy, such forces may mobilize implants.

This fact must be considered especially in patients with a skeletal Class II jaw relationship that must be restored with this relationship persisting. It is just this patient group that has only unreliable control over the masticatory forces exerted. The further the anomaly has progressed, the more pronounced the anterior masticatory pattern usually found in these patients, a pattern that forces the temporomandibular joint into wide protrusive movements.

Also, in many cases the temporomandibular joint is relocated dorsally once the support zone is lost. The positional variability of the temporomandibular joint makes it impossible to determine jaw relations with any degree of certainty in these patients, and unilateral premature contacts must often be corrected in the early phases of functional use. Since anterior support is often absent, 100% of the occlusal forces will occur in the distal maxilla, where they meet the bone that is initially weakest.

Implant healing without loading, demanded by the manufacturers of many systems, requires direct interaction between the implant surface and the surrounding bone to be the major factors of implant integration. No additional stimuli derived from masticatory function, which might give the spongious and cortical bone areas direction and maturity, are present in this treatment protocol. At the same time, the masticatory force after implantation without early implant loading will be unchanged at best, which is why osteotomy-related remodelling results in a reduction of overall bone volume.

Especially the lateral maxilla contains bone areas with low mineralization activity. The maxillary sinus usually expands, while the vertical bone supply is reduced from the oral side. Bone quality according to Misch is often only D3 or D4.

The reason for this development in the areas mentioned is that the region between the first upper premolar and the second upper molar is primarily subjected to tensile forces. The tension is created by the forward pressure of the mandible onto the anterior teeth and bone regions and the distomedial tensile force of the lateral pterygoid muscle. Thus, both the anterior and the far distal areas of the maxilla are well-stimulated areas (1-areas). The area in between is a zone of predominant tension with consequent lower mineralization (0-area).

Compressive forces exerted by the tongue affect only the palatal side, which is why the bone is preserved longer in that area. By contrast, the bone is not preserved or only preserved to a lesser extent where net tensile forces act on it. Bone loss in itself is direct evidence of the presence of such tensile forces. (From the usual vantage point of our profession, this relationship is difficult to comprehend, as we have traditionally learned that pressure, not tension, causes bone loss, especially below dentures.)

If implants are placed in tensile areas, this in itself creates particularly unfavourable conditions for the implants themselves and especially for immediate loading. To avoid detachment of the bony interface from the implant and overload in areas that had been subjected only to minor load preoperatively, the reduction of masticatory forces appears to be a sensible therapeutic adjunct.

Once the implant has healed, the masticatory forces that were transmitted enossally are incentive enough to permit sustained osseointegration with a high degree of mineralization even in previously tensile areas.

The potential micromorphological consequences of the reduction in masticatory forces acting on the bone must also be discussed. If the load is reduced and with it the elastic deformation of osteonal bone, stasis occurs in the osteonal transport system, and increased remodelling is subsequently seen. While porosity increases, the mineral level of the affected region gradually decreases over several weeks, optimizing bone elasticity. That this does not lead to the complete disappearance of the jawbone in the implanted areas is a result of the masticatory forces still reaching the peri-implant bone and stimulating these areas as a result of immediate loading. By contrast, macrotrajectorial loads that would generate a different, more globally oriented pattern of mineralization are greatly reduced. This is why it is probably safer to implant in 0-areas when the muscles generating the global mineralization pattern are weakened or temporarily incapacitated.

It is within the scope of the present invention that this medication can be used as an adjunct in treatment concepts including either crestal implants or a combination of crestal and basal implants, as well as basal implants alone.

Botulinum toxin may be advantageously used in prophylactic application, especially in cases with reduced bone quantity and quality and in cases of immediate loading in compromised bone situations. The phase of functional use of enossally supported bone/implant/restoration systems is often characterized by extensive changes in the relative positions of the dental arches, segments or individual teeth. The extent and sequence of these changes cannot be predicted, which is why the inserted restorations must be monitored and adjusted at regular intervals. Even more unpredictable are the morphological changes—which can have a variety of causes—in the implanted jawbone, changes that the integrated implants and thus the functional surfaces of the restorations will passively follow. In addition to masticatory force and masticatory function, age, hormonal status and genetic dispositions as well as habits and other factors will play a role in determining the nature and extent of these changes, even beyond the practitioner's experience with tooth-supported restorations.

The peri-implant bone can be affected in two different ways—independently of causation—with patients also potentially experience both of them consecutively:
(1) If the bone/implant interface is overloaded, the bone and implant may separate, resulting in a loss of positive retention. On the other hand, overload may result in cumulative microcracks and subsequent sterile overload osteolysis.
(2) If regional tensile forces dominate on the underload side, the implants will become mobile despite continued osseointegration, since the load-bearing capacity of the bone bed in itself has changed due to increased in the porosity of the bone. On the underload side, even smaller masticatory forces that had previously been well tolerated may cause or promote overload-related osteolysis at the bony interface as described under (1).

The masticatory forces will return to previous levels once the effect of the drug has subsided, possibly once again exercising their deleterious functions. However, a permanent reduction of masticatory forces is not the therapeutic objective; rather, the objective is to create a more favourable load situation during a phase of higher elasticity in the region of the bony interface for a limited time, namely the time it takes for the bone to remineralize and the implant to reintegrate in the bony interface region. Unless premature contacts and unilateral loading were not addressed at the outset of botulinum toxin therapy, the stabilization of the bone/implant system may not be a lasting success as the causes of instability would persist.

Botulinum toxin therapy reduces masticatory forces to a level where they once again constitute an impetus for the creation of more highly mineralized interfacial bone even in regions of increased elasticity. At the same time, more global trajectories of higher mineralization will have stress removed from them, potentially resulting in increased remodelling in these areas and in redistribution of 0-areas and 1-areas. When the masticatory forces return in due course, the degree of mineralization of the jawbone will increase again. Bone quality as measured in Hounsfield units is thus more or less a measure of the extent to which the bone is predominantly trained and loaded prior to the clinical examination and subsequent therapy and not any indication on potential performance after implantation and functional loading. It seems generally advisable not only to perform histological measurements of the bone interface within the framework of scientific studies of the integration behaviour of implants of all types but also to determine the degree of mineralization in the interfacial region.

Histological studies have been able to show that a higher degree of integration is gradually reached during the phase of actual function of lateral implants. Several different strategies are available to the bone: Lateral excursive movements of the threaded pin result in bone apposition in the vertical implant regions. Within the bone, the bone adaptation region can be increased by gap jumping, by osseoadaptation or by an orientation of the bone mediated by force-oriented connective tissue.

Clinicians should note that anterior contacts generally increase in number due to a slight anterior dislocation of the mandible if only the masseter muscles are medicated. This change has to be accommodated by timely small adjustments of the occlusal surfaces. Sufficient free anterior space must exist for the mandibular anterior teeth, or space has to be created through the mandibular posterior build-up.

In addition, the muscles will shorten as their activity increases with the recovery of masticatory forces once the toxin is no longer effective. This means that the distal aspects of the superstructure between which a correct vertical relationship had existed while the muscles were not active, need to be reduced in a timely manner to avoid excessive loading.

In one embodiment of the invention, bilateral medication of the masseter muscles will generally suffice. If the temporal muscles are particularly prominent clinically or if jawbone atrophy is unusually strong, this limited use of botulinum toxin might certainly be considered, with precedence probably given to treating the two large masticatory muscles. However, it is within the scope of the present invention that parts of the temporal muscles should be injected as well.

Reintegration of enossal implant surfaces can not be expected if infection occurs which cause granulomatous changes in the bony interface. Botulinum toxin therapy may be used with crestal implants (i.e. screw-type implants). The greater amount of micro-roughness on the vertical interfaces of later allows infections to get inoculated by vertical movements and advance rapidly, thereby in theory reducing the chance of reintegration as described above, even if the masticatory load is reduced.

The forces exerted by the large masticatory muscles can be temporarily reduced by the application of botulinum toxin (Dysport®) to the masseter muscle. This is a possibility to increase the safety of treatment procedures in dental implantology and to extend the indications for immediate-loading treatment protocols at the same time. The use of the drug appears to be particularly beneficial if large masticatory forces are known to exist, weak and osteoporotic bone is encountered during the implant procedure, and if parafunction is feared or has been experienced anamnestically. Additional indications exist in our view, if the total bone supply is reduced and if the restorative treatment of Class II jaw relations must be implemented in the absence of circular support. Given these options, implant treatment with immediate loading of implants is possible.

### CONCLUSION

Botulinum toxin can reduce the indirect influence of the masticatory load on the bone/enossal implant interface. If undesirable loads distributions between the occlusal surfaces of both jaws are eliminated, the use of botulinum toxin on the large masticatory muscles can create an environment that is favourable to the reintegration of implants and to bone remineralization. Botulinum therapy targets the balance between demineralization and remineralization. The chances of the therapy with botulinum toxin may make it easier to decide against removing an ailing (lateral) implant and favours steps for preserving an implant/restoration system that is not entirely stable clinically due to masticatory overload.

Other objects, features and advantages will be apparent to those skilled in the art. The invention in its broader aspects is not limited to the specific steps and embodiments shown and described but departures may be made therefrom within the scope of the appended claims without departing from the principles of the invention and without sacrificing its chief advantages.

What is claimed is:

1. A method of stabilizing masticatory forces around dental implants, the method comprising:
installing medical hardware in a patient's mouth;
calculating a dose of botulinum toxin; and
injecting said dose of botulinum toxin into a masticatory muscle of said patient.

2. The method of claim 1 wherein said dose of botulinum toxin corresponds to a preconfigured portion of a time for healing.

3. The method of claim 1 wherein said calculation step associates said dose with parameters selected from the group consisting of:
a patient's age,
an estimate of muscle strength;
a position of the dental implant;
a diet of the patient;
a number of implants;
a location of the botulinum toxin injection;
a presence of nocturnal parafunction;
a amount of bone into which the implant has been installed; and
a nature and quality of the bone into which the implant is being installed.

4. The method of claim 1 wherein said medical hardware is at least one dental implant.

5. The method of claim 4 wherein said dental implant is a basal implant.

6. The method of claim 1 wherein said method is used to treat nocturnal parafunction.

7. A system for treating masticatory pathology comprising:
providing a hardware set, said hardware set being configured to be installed in a patient's mandible or maxilla and to cure said masticatory pathology;
associating said installation of said hardware set with an anticipated healing time;
correlating a dose of botulinum toxin with a portion of said anticipated healing time for each of said anticipated healing times;
providing a supply of said botulinum toxin sufficient to comprise said dose; and
providing a hypodermic needle to inject said dose of botulinum toxin.

8. The system of claim 7 wherein said system is used to remediate a complication of a previous installation of said hardware set.

9. The system of claim 7 wherein said dental implants include crestal implants.
